(19) United States
(12) Patent Application Publication
(10) Pub. No.: US 2024/0358772 A1
(43) Pub. Date: Oct. 31, 2024

(54) Mycobacteria From Single Colony Type for Vaccine and Other Immunotherapy

(71) Applicant: S & R Pharmaceuticals, Lenox, MA (US)

(72) Inventor: Tsungda Hsu, Lenox, MA (US)

(21) Appl. No.: 18/307,297

(22) Filed: Apr. 26, 2023

(51) Int. Cl. A61K 35/741; C12N 1/20

(52) U.S. Cl. CPC A61K 35/741; C12N 1/20

(57) ABSTRACT

A method of making a therapeutic Mycobacteria composition. Live Mycobacteria (e.g. M. bovis) that have a genetic trait for dimorphic colony formation are made into a single cell suspension. The Mycobacteria suspension is spread onto a non-liquid culture medium. The Mycobacteria grows such that it forms colonies of both R-type and S-type morphologies. Mycobacteria from an S-type colony is harvested and processed to create the therapeutic Mycobacteria composition. The therapeutic composition lacks any Mycobacteria that grew from an R-type colony. The culture medium may lack polysorbate-80 to promote growth as S-type colonies. The Mycobacteria could have an alteration to its genome that impairs growth of the Mycobacteria.

### TECHNICAL FIELD

This invention relates to Mycobacteria for immunotherapy vaccines.

### BACKGROUND

Tuberculosis (TB) is an infectious disease caused by Mycobacterium tuberculosis. Many years ago, a vaccine was developed for tuberculosis disease. This vaccine is called Bacillus Calmette-Guérin (BCG) and was first used in humans in 1921. BCG is an attenuated strain of Mycobacterium bovis, which is a related species. Because the efficacy of BCG in preventing tuberculosis varies, it is not as widely used as it should. However, BCG has found another use in the treatment of bladder cancer. More recently, BCG vaccination could be helpful against Covid-19 (SARS-COV-2 infection).

Mycobacterium tuberculosis have remarkable ability to survive within the host. One of the major mechanisms of resilience by Mycobacteria is having subpopulations with distinct phenotypes that can adapt to changes in the host environment. This heterogeneity is a vital survival strategy. One type of Mycobacteria heterogeneity is different colony morphologies, in particular, switching colony morphology between smooth (S) and rough (R) forms. Colony morphology changes can be mediated by genetic variations.

Further background information about the invention is given in the Appendix to the Specification, which contains a manuscript titled “An Inducible Phase Variation System in Mycobacterium Tuberculosis” (Appendix A) and the main body of a grant application (Appendix B). These items contain bibliographic references that may be relevant to this invention.

### SUMMARY

This invention encompasses a therapeutic Mycobacteria composition, medical products that can be made therefrom, medical therapies using such, methods of making such, etc. Composition. In one aspect, this invention is a therapeutic Mycobacteria composition. It comprises a population of live, but attenuated Mycobacteria. In some embodiments, all the Mycobacteria in the therapeutic composition have a trait for dimorphic colony growth. That is, the population of Mycobacteria in the therapeutic composition consists essentially of those that are able to grow into dimorphic colonies of both S-type and R-type. The Mycobacteria have one or more alterations to its genome that causes it to exhibit this trait for dimorphic colony growth. Various genomic loci and types of alterations may impart this trait. In some cases, the Mycobacteria have a genome alteration that makes it become auxotrophic.

In some embodiments, the source of the Mycobacteria in the therapeutic composition is from one or more colonies of a single morphology type (S-type or R-type) that are grown from a suspension of single cells that are spread on the surface of a non-liquid medium substrate. In the case of an S-type Mycobacteria composition, the Mycobacteria composition has only Mycobacteria that grew from an S-type colony. This Mycobacteria composition essentially lacks Mycobacteria that grew from an R-type colony. In the case of an R-type Mycobacteria composition, the Mycobacteria composition has only Mycobacteria that grew from an R-type colony. This Mycobacteria composition essentially lacks Mycobacteria that grew from an S-type colony.

The Mycobacteria may be capable of stably maintaining the dimorphic colony growing trait through subsequent generations. In some embodiments, the Mycobacteria have a trait for only S-type colony growth. The therapeutic composition may be in dry powder form (e.g. lyophilized). The therapeutic composition may further comprise other excipient ingredients that could serve as stabilizers, buffering agents, surfactants, antioxidants, preservatives, etc. Examples of such excipient ingredients include glycerin, asparagine, citric acid, potassium phosphate, magnesium sulfate, iron ammonium citrate, sodium glutamate, etc.

In some embodiments, this invention could be defined as a therapeutic Mycobacteria composition consisting essentially of a population of live, attenuated Mycobacteria and optionally, one or more excipient ingredients. In some cases, the Mycobacteria grew (only) from an S-type colony. In some cases, the Mycobacteria grew (only) from an R-type colony. In some embodiments, the therapeutic composition lacks any Mycobacteria that grew from an R-type colony. In some embodiments, the therapeutic composition lacks any Mycobacteria that grew from an S-type colony.

Medical Product. In another aspect, this invention is a medical product comprising the therapeutic Mycobacterium composition. The product comprises a dispensing container that contains the therapeutic Mycobacterium composition. The product may be single-use or multi-use. The dispensing container may be any suitable type of container for holding medical compositions, such as bottle, vial, or ampoule.

The amount of therapeutic Mycobacterium composition in the dispensing container may be for sufficient for single or multiple doses. The amount could depend on the medical use. In some embodiments, the dispensing container contains 5-100 mg (wet weight, hereafter) of the therapeutic Mycobacterium composition. In some embodiments, the dispensing container contains 1×10⁴-1×10⁶ cfu (colony forming units) of the therapeutic Mycobacterium composition. These amounts may be useful for vaccination against tuberculosis disease.

In some embodiments, the dispensing container contains 15-150 mg (wet weight) of the therapeutic Mycobacterium composition. In some embodiments, the dispensing container contains 1×10⁷-1×10¹⁰ cfu (colony forming units) of the therapeutic Mycobacterium composition. Other amounts (as explained below) could be 1×10³-1×10⁸ cfu of the therapeutic Mycobacterium composition; or 1×10³-1×10⁵ cfu; or 1×10⁶-1×10⁹ cfu; or 1×10⁵-1×10⁷ cfu. Other amounts (as explained below) could be 10-40 mg (wet weight) of the therapeutic Mycobacteria composition; or 10-30 mg.

Medical Method: The therapeutic Mycobacteria composition may be used for any suitable type of therapeutic use in humans or animals, such as treatment, prophylaxis, or maintenance regimen. Examples of therapeutic uses include vaccination against tuberculosis disease (TB), protection against SARS-COV-2 infection (virus responsible for Covid-19), immunotherapy against cancer (e.g. bladder cancer), etc. In another aspect, this invention is method of providing immune activating medical therapy to a patient. The method comprises administering the therapeutic Mycobacteria composition to the patient. The therapeutic composition may be administered in any suitable way. If in powder form, the therapeutic composition may be made into liquid form by mixing with an aqueous solution (e.g. water or saline). For example, for treatment of bladder cancer, the therapeutic composition may be instilled into the patient's bladder (intravesically by catheter). In another example, for vaccination against tuberculosis disease, the therapeutic composition may be administered into the patient's skin (e.g. intradermal injection or by a multipuncture device).

The therapeutic Mycobacteria composition may elicit a T-cell response in the patient when administered. In some embodiments, the therapeutic composition causes a Th2 dominant response when administered to the patient. In some embodiments, the therapeutic composition causes a Th1 dominant response when administered to the patient. A Th1 response may be particularly useful for immunotherapy against cancer, such as bladder cancer.

The dosing amount may depend on various factors such as the type of medical therapy and potency of the therapeutic Mycobacteria composition. In some embodiments, the amount of the therapeutic composition administered to the patient is 15-150 mg (wet weight). Because this therapeutic composition contains only a single type of Mycobacteria, it may produce a more potent effect than the conventional BCG Mycobacteria product. As such, less of the therapeutic Mycobacteria composition of this invention may be required to achieve the same or better efficacy compared to BCG Mycobacteria.

For vaccination against tuberculosis disease, the patient may be administered 1×10³-1×10⁷ cfu of the therapeutic composition; and in some cases, 1×10³-1×10⁵ cfu. For vaccination against tuberculosis disease, the patient may be administered 10-40 mg (wet weight) of the therapeutic Mycobacteria composition; and in some cases, 10-30 mg. For treating cancer, the patient may be administered 1×10⁵-1×10¹⁰ cfu of the therapeutic composition; and in some cases, 1×10⁶-1×10⁸ cfu. For treating cancer, the patient may be administered 10-40 mg (wet weight) of the therapeutic Mycobacteria composition; and in some cases, 10-30 mg.

Method of Making: In another aspect, this invention is a method of making the therapeutic Mycobacteria composition. In some embodiments, use Mycobacteria that have a genetic trait for dimorphic colony formation. Make a single cell suspension of the Mycobacteria in a suspension fluid. Dilute the Mycobacteria suspension if needed. Spread the Mycobacteria suspension onto the surface of a non-liquid culture medium. Incubate to grow the Mycobacteria such that it forms colonies of both R-type and S-type morphologies on the culture medium. To promote dimorphic colony formation, the culture medium may lack polysorbate-80 or have a low content thereof.

Depending on which type of Mycobacteria is desired for the therapeutic composition, harvest the Mycobacteria from only an S-type colony (one or more thereof), from only an R-type colony (one or more thereof), or both separately. In some embodiments, identify an S-type colony and transfer Mycobacteria therefrom to a suspension fluid. Create a single cell suspension of the Mycobacteria. The Mycobacteria in the suspension fluid could be filtered or diluted to help create the single cell suspension. In some embodiments, identify an R-type colony and do the same to create a single cell suspension of the Mycobacteria. Process the harvested Mycobacterium to create a therapeutic Mycobacteria composition of this invention. This processing may involve any of various conventional steps used in making biologic pharmaceuticals, such as emulsifying, lyophilizing, packaging, mixing with excipient ingredients, etc.

In some embodiments, use Mycobacteria that have a genetic trait for only S-type colony growth. Perform the same steps as above for making a single cell suspension and growing the Mycobacteria such that it forms colonies of only S-type morphology. To promote only S-type colony formation, the culture medium may lack polysorbate-80 or have a low content thereof. Harvest Mycobacteria from an S-type colony (one or more thereof) and process in the same manner as above to create a therapeutic Mycobacteria composition of this invention.

### BRIEF DESCRIPTION OF THE DRAWINGS

FIGS. 1A-1C show an example of Mycobacteria growing on an agar plate as dimorphic colonies. FIG. 1A shows the overall view. FIG. 1B shows a closeup view of an S-type colony. FIG. 1C shows a closeup view of an R-type colony.

FIG. 2 shows an example of Mycobacteria growing into pleiomorphic colonies.

FIG. 3 shows an example medical product as a vial containing a therapeutic Mycobacteria composition of this invention.

FIG. 4 shows an example of bladder cancer being treated with the Mycobacteria composition of this invention.

### DETAILED DESCRIPTION OF EXAMPLE EMBODIMENTS

Terminology used herein are as follows. The Mycobacteria used in this invention may be any of the various species such as M. tuberculosis, M. smegmatis, M. bovis, M. avium complex, chelonae, M. phlei, M. abscessus, etc. “Trait for dimorphic colony formation” means that the Mycobacteria will grow into colonies of both S-type and R-type, as tested when grown from a single parent cell on the surface of a solid media substrate upon transfer by direct plating technique. “Trait for only S-type colony growth” means that the Mycobacteria will grow into colonies of only S-type morphology, as tested when grown from a single parent cell on the surface of a solid media substrate upon transfer by direct plating technique.

“Direct plating technique” means subculturing a Mycobacteria colony from a non-liquid culture medium by sampling bacterial cells from the colony, mixing the sample of bacterial cells with a suspension fluid, making this into a single cell suspension of the Mycobacteria, spreading the single cell suspension onto a non-liquid culture medium, and incubating for growth of the Mycobacteria. This is done without any intervening step of growing the Mycobacteria in a nutrient broth. Making the single cell suspension is not for growing the bacteria. The suspension fluid is merely intended as a transfer medium for single cell processing. As such, the Mycobacteria reside in the suspension fluid for a duration of less than two hours; and in some cases, less than one hour. Single cell processing may further involve filtration or dilution to help obtain single cell units.

The Mycobacteria have the trait for dimorphic colony growth (or trait for only S-type colony growth) because of one or more alterations to its genome. These alterations may be any type conventionally used or found in mutations, such as substitution, insertion, deletion, duplication, inversion, frameshift, repeat expansion, etc. The genomic loci where the alteration occurs may have any function, such as coding genes, non-coding DNA, operon, or regulatory sequences (e.g. promoter, repressor, activator, silencer, enhancer, etc.). The genetic trait could be an alteration in one or more genomic loci that causes the Mycobacteria to become auxotrophic.

“Auxotrophic” means that the Mycobacteria is unable to synthesize a particular organic compound required for its growth, as defined by IUPAC (International Union of Pure & Applied Chemistry). For example, M. tuberculosis with the leuCD deletion mutation are unable to synthesize leucine. Thus, they require leucine as a supplement in the culture medium for sustained growth.

“Stably maintain the dimorphic colony trait” means that the Mycobacteria are capable of maintaining the trait for dimorphic colony formation through at least a first generation colony descended from a single parent cell taken from the parent colony; and in some cases, at least into a second generation colony descended from a single parent cell taken from the first generation colony. Further, a high proportion of S-type colonies may be maintained in those descendant generations. In some cases, at least 30% of the total colonies that grow in a subsequent generation are S-type; and in some cases, at least 40%.

“Colony” means a visible mass of Mycobacteria growing on a solid substrate that originate from the same single parent cell or small clumps thereof. Visible characteristics of bacterial colonies include size, shape (e.g. round, irregular, filamentous, rhizoid, etc.), elevation above substrate, surface texture (e.g. smooth, rough, wrinkled, etc.), color, opacity, and shine (glistening, dull, etc.).

R-type colonies on the surface of a culture substrate generally have the following visible characteristics compared to S-type colonies: relatively larger, round shape, spreading, rough surface texture, a fried-egg center, and raised only slightly above the substrate surface. All these features are not required for a Mycobacteria colony to be considered R-type.

S-type colonies on the surface of a culture substrate generally have the following visible characteristics compared to R-type colonies: relatively smaller, irregular shape, confined, smooth surface texture, absence of fried-egg center, and distinctly raised from the substrate surface. All these features are not required for a Mycobacteria colony to be considered S-type.

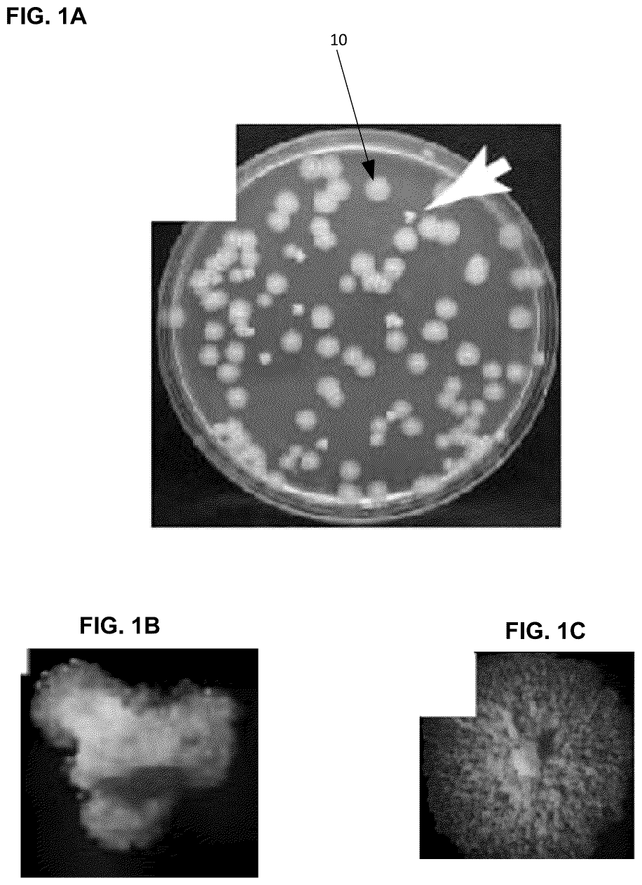

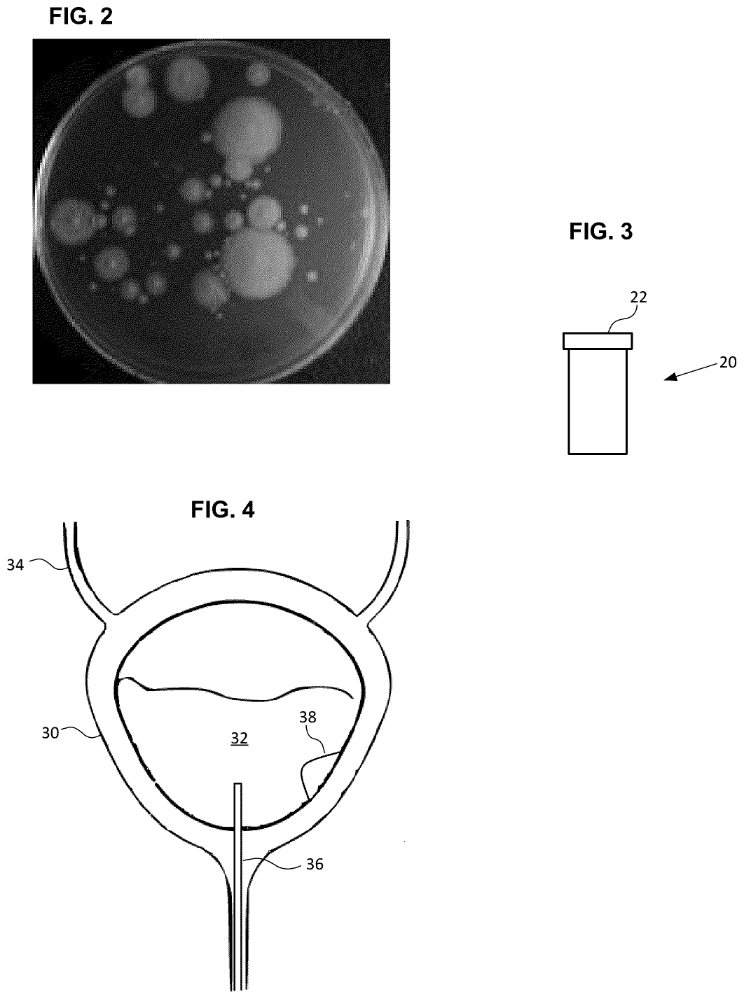

Both S-type and R-type colonies are of uniform size and shape with respect to their own morphology. This is different from pleiomorphic colonies, which have three or more different morphology types with different sizes or shapes. FIGS. 1A-1C show an example of Mycobacteria growing on an agar plate as dimorphic colonies. FIG. 1A shows the overall view. The white arrowhead points to an S-type colony. Arrow 10 points to an R-type colony. FIG. 1B shows a closeup view of the S-type colony at the white arrowhead. FIG. 1C shows a closeup view of the R-type colony at arrow 10. FIG. 2 shows an example of Mycobacteria growing into pleiomorphic colonies.

“Suspension fluid” means any suitable liquid fluid that can be used to hold Mycobacteria cells alive while they are being processed into a suspension of single cells. Examples of suspension fluids include phosphate-buffered saline (PBS), other buffer solutions, liquid nutrient broths, etc.

“Single cell suspension” means that at least some of the Mycobacteria in the volume of suspension fluid are dissociated into individual single cells. This does not necessarily mean that all the cells therein are dissociated into single cell units. There may still be some cell aggregates in the suspension, such as small clumps of cells. In some cases, at least 50% of the Mycobacteria cells in the suspension are dissociated into individual single cells.

“Attenuated” means substantially reduced human virulence compared to wild-type such that it has a clinical safety profile that meets the vaccine regulatory approval criteria of the “WHO Guidelines on Clinical Evaluation of Vaccines” (2001), or “Guideline on Clinical Evaluation of New Vaccines” (Jan. 2023) by the European Medicines Agency, or similar guidelines issued by the U.S. Food & Drug Administration, or successor guidelines thereof. In particular, “attenuated” may mean having the same or lower level of human virulence compared to M. bovis Pasteur (BCG). “Live” means that the bacteria are capable of growing under standard in-vitro culture conditions.

In some embodiments, the culture medium is essentially free of polysorbate-80 (also known as polyoxyethylene-80 sorbitan mono-oleate, e.g. Tween 80) or have a low content thereof. “Low content” means less than 0.01% content thereof, as measured by weight/weight of the solid matter. “Essentially free” is not intended to exclude any incidental amounts that might be present in commercial products (despite the absence of polysorbate-80 on the product literature) or trace amounts that cannot be eliminated by conventional methods.

“Non-liquid culture medium” means bacterial growth medium that is solid or semi-solid. An example of a liquid medium is nutrient broth. An example of a solid medium is a slab of 1-2% agar (w/w) in a culture dish. Semi-solid medium has a lower solid content. An example of semi-solid medium is 0.2-0.5% agar gel in a culture dish. For solid or semi-solid culture medium, the bacteria may be grown on the surface of the substrate medium.

Helper T lymphocytes (also known as helper T-cells) can activate two different types of responses: Th1 and Th2. Th1 response is characterized by secretion of one or more of the following proinflammatory cytokines by Th1-type helper lymphocytes (T-cell): IFN-γ, TNF-α, IL-2, TNF-β (referring to interferon-gamma, interleukin, and tumor necrosis factor). These activate CD8+ cytotoxic T-cells against intracellular pathogens. Th2 response is characterized by secretion of one or more of the following cytokines by Th2-type helper lymphocytes (T-cell): IL-4, IL-5, IL-10, IL-13 (referring to interleukin). These activate B-cells and antibody production.

Mycobacteria harvested from an S-type colony may elicit a Th1 dominant response, whereas Mycobacteria harvested from an R-type colony may elicit a Th2 dominant response. “Th1 dominant response” means that the above-mentioned characteristics of the Th1 response are dominant over the above-mentioned characteristics of the Th2 response. For example, a response characterized by a large increase in serum IFN-γ levels would be a Th1 dominant response. “Th2 dominant response” means that the above-mentioned characteristics of the Th2 response are dominant over the above-mentioned characteristics of the Th1 response. For example, a response characterized by IgE antibody production and eosinophilia would be a Th2 dominant response.

“Excipient ingredient” means a substance that is not the Mycobacteria, but is added to the composition to facilitate product formulation, such as improving handling, storage, stability, dispersion, emulsification, etc.

FIG. 3 shows an example of vial 20 containing a single dose amount of the therapeutic Mycobacteria composition in lyophilized powder form. The vial 20 is sealed with a cap 22. FIG. 4 shows an example of bladder cancer being treated with a Mycobacteria composition of this invention. A catheter 36 is inserted into the bladder 30. The Mycobacteria composition 32 (suspended in saline water) is instilled into the bladder 30 via the catheter 36. Also shown is the bladder cancer tumor 38 and the pair of ureters 34 going into the bladder 30.

### EXPERIMENTAL WORK

The following is a condensed and summary description of the experimental work showing that deletion of the RD1 genomic locus induced M. tuberculosis to produce both R-type and S-type colonies (dimorphic colony trait). This is in contrast to wild-type M. tuberculosis that grow only as R-type colonies. This indicates that the RD1 locus harbors genetic element(s) mediating M. tuberculosis ability to undergo colony dimorphism (by phase variation). More detailed reporting of the experimental work is in Specification Appendix A (manuscript for journal submission).

Bacterial Strains & Culture. Unless otherwise indicated, the various Mycobacteria strains were cultured using previously published standard media and culture conditions. This includes using 7H9 (Middlebrook) broth media, 7H10 solid agar media, supplementation with Tween 80 (to prevent the formation of large aggregates) or OADC, agar plating in petri dishes, and incubating at 37° C.

Colony Dimorphism by RD1 Deletion. As the main comparative, culture of non-filtered M. tuberculosis H37Rv growing in 7H9T broth media and spread onto 7H10 agar plates produced the conventional R-type colonies in uniform size and shape. As a candidate to explore inducement of colony dimorphism, select M. tuberculosis H37Rv with deletion of the RD1 genomic locus. Make this mutation strain using a 2-step sequential homologous recombination method as previously described. Grow M. tuberculosis H37Rv as R-type colonies. Transform the bacteria by electroporation with plasmid pJH508 which harbors an allelic exchange substrate for RD1. Recombination occurs in the downstream homologous sequences between pJH508 and the bacterial genome. Screen colonies for the RD1 deletion by southern blot analysis. This deletion strain is designated as mc²4002.

When growing the mc²4002 for testing, ensure that they are free of contamination by M. tuberculosis H37Rv. For this, prepare single-cell suspensions of mc²4002 by growing them in 7H9T broth media, and passing the liquid sample through 5 μm filter. The resulting filtered suspensions contain mostly single bacterial cells or small aggregates. Dilute the filtered suspension, spread on 7H10 agar plates, and incubate for 4-6 weeks.

Surprisingly, the mc²4002 grew into two distinct types of colonies. Some of them grew into R-type colonies. They appeared as large and spreading with a fried-egg center, similar to wild-type M. tuberculosis H37Rv. Others grew into S-type colonies. They were small, non-spreading, and on some occasions, exhibited a smooth surface texture. Also surprisingly, DNA analysis showed that both (not just one) S-type and R-type colonies carried the RD1 deletion. To determine if RD1 harbored genetic elements that are responsible for the colony dimorphism, mc²4002 was complemented with an RD1 cosmid 2F9, which integrated at the att-L5 site of the genome of M. tuberculosis H37Rv. This resulted in the generation of strain mc²4019 which produced only R-type colonies on 7H10 agar plates (reversion).

Pleiomorphic Colonies. Producing dimorphic colonies of Mycobacteria is challenging. One major challenge is that pleiomorphic colonies can result, instead of dimorphic colonies of S-type or R-type in uniform size and shape. Other culture techniques can often result in pleiomorphic colonies instead of dimorphic colonies. As one example, single cell suspensions prepared directly from M. tuberculosis H37RV thawed from frozen stocks and spread on 7H10 or 7H10T agar plates produced colonies of different sizes and shapes (pleiomorphism). In another example, single-cell suspensions of M. tuberculosis H37Rv mc²4000 (merodiploid, RD1::pJH508) spread on 7H10 agar plates produced colonies of uniform size and shape. But spreading the same single-cell suspensions on 7H10 agar plates supplemented with 0.05% Tween 80 produced colonies of at least three differences sizes. Some were small colonies whereas others grew into large colonies; and some made even larger colonies. This pleiomorphism was likely caused by the presence of Tween 80 in the substrate media.

Other Genetic Disruptions. Disruption of the following Mycobacterium genomic loci have also been demonstrated to impart the trait for dimorphic colony growth: Rv3879 (such as pJH508 plasmid insertion therein); esxB (such as insertion of Tn5370 therein); Rv0007, Rv0008c, Rv0010c, Rv0011c, Rv0023, Rv0025, Rv0026, Rv0028, Rv0030, Rv0051, Rv0057, Rv0072, Rv0078, Rv0081, Rv0082, Rv0083, Rv0086, Rv0090, Rv0096, Rv0109, Rv0139, Rv0342, Rv0343, Rv0379, Rv1246c, Rv1848, Rv1849, Rv1860, Rv1908c, Rv1909c, Rv2057c, Rv2866, Rv3578, Rv3874, and Rv3875.

These genomic loci have a wide variety of different functions. But the common feature is that disruption in these genomic loci impair the Mycobacteria growth. Thus, any alteration to its genome that impairs growth could impart the trait for dimorphic colony growth (or the trait for only S-type colony growth). This is because the Mycobacteria undergo phase variation when they sense that growth is being retarded. By having different members of its population convert to different colony forms, this increases the diversity of characteristics. The Mycobacteria seeks to diversify its population by phase variation as an attempt to find a phenotype more suitable for the growth retarding conditions. For example, the more compact S-type morphology may be an adaptation for improved survival in such situations.

Other Observations. Other experimental observations are as follows. • Whereas wild-type M. bovis Ravenel grew only as R-type colonies, M. bovis Ravenel ΔRD1 produced both R-type and S-type colonies on 7H10 agar plates. • Sometimes, S-type colonies are enclosed (completely or partially) by larger R-type colonies. However, mixed or sectored colonies displaying both S-and R-type characteristics were never observed. As soon as an incipient colony was barely visible, it stably maintained one morphology type. This implies that the morphology type is “locked in” once a colony begins to form. • In a study of gene transcriptional profiles by microarray analysis, samples from the S-type colonies exhibited upregulation of genes important for hypoxic conditions, stress conditions, or intracellular growth. Samples from the R-type colonies exhibited upregulation of genes important for aerobic growth.

M. bovis Ravenel could be transduced with the RD1 specialized transducing phage to generate the RD1 deletion mutant. • Whether the source of the cells is directly from broth cultures or from agar plates makes a difference in morphology type conversion ratio. Diluted samples of single cell suspensions of BCG Pasteur cultured in 7H9T broth and then spread onto 7H10 agar plates grew R-type colonies at a frequency of 25-79%. However, diluted samples of the same single cell suspensions prepared from harvested colonies of S-type and R-type grown on agar plates (instead of broth media) by direct plating technique, and then spread onto 7H10 agar substrate, grew into both types at about 50% ratio. • The ability to grow into dimorphic colonies was stably maintained into subsequent generations. First and second descendant generations that were grown directly from parents that were harvested from agar plates by direct plating of single cell suspensions grew into dimorphic colonies at about 50% ratio (of S-and R-type). • There was one M. tuberculosis ΔRD1 strain that grew only as S-type colonies. Further investigation of this strain is needed. But this demonstrates that making Mycobacteria having a trait for only S-type colony growth is possible. • Vaccine injection of S-type BCG Mycobacteria into mice triggered cytokines indicating a strong Th1 response.

The descriptions and examples given herein are intended merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

The terms “first, second, etc.” with respect to elements may be used herein only to distinguish one element from another element. Unless the context indicates otherwise, these are not intended to limit the elements regarding their composition or ordinal arrangement, such as defining the order, position, or priority of the elements. Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A method of making a therapeutic Mycobacteria composition, comprising:
having live Mycobacteria that have a genetic trait for dimorphic colony formation;
making a single cell suspension of the Mycobacteria in a suspension fluid;
spreading the Mycobacteria suspension onto a surface of a non-liquid culture medium;
growing the Mycobacteria such that it forms colonies of both R-type and S-type morphologies on the culture medium;
identifying an S-type colony and harvesting Mycobacteria therefrom;
processing the Mycobacteria to create the therapeutic Mycobacteria composition;
wherein the therapeutic composition lacks any Mycobacteria that grew from an R-type colony.

2. The method of claim 1, wherein processing the Mycobacteria comprises lyophilizing the Mycobacteria.

3. The method of claim 1, wherein the Mycobacteria in the therapeutic composition are only from an S-type colony.

4. The method of claim 1, wherein the suspension fluid is a first suspension fluid, and processing the Mycobacteria comprises suspending the harvested Mycobacteria in a second suspension fluid.

5. The method of claim 1, wherein the non-liquid culture medium lacks polysorbate-80 or has a low content thereof.

6. The method of claim 1, wherein the steps of making a single cell suspension of the Mycobacteria and spreading the Mycobacteria suspension onto the surface of the non-liquid culture medium is performed by direct plating technique.

7. The method of claim 1, wherein the therapeutic Mycobacteria composition is a first therapeutic Mycobacteria composition, and the method further comprises:
identifying an R-type colony and harvesting Mycobacteria therefrom;
processing the Mycobacteria to create a second therapeutic Mycobacteria composition;
wherein the second therapeutic composition lacks any Mycobacteria that grew from an S-type colony.

8. The method of claim 1, wherein the Mycobacteria does not grow into pleiomorphic colonies on the non-liquid culture medium.

9. The method of claim 1, wherein at least 30% of the colonies that grow on the non-liquid culture medium are S-type colonies.

10. The method of claim 1, wherein the Mycobacteria has an alteration to its genome that imparts the trait for dimorphic colony growth.

11. The method of claim 10, wherein the genome alteration impairs growth of the Mycobacteria.

12. The method of claim 10, wherein the genome alteration is a disruption in an RD1 locus.

13. The method of claim 1, wherein the Mycobacteria maintains the dimorphic colony trait for at least a first descendent generation.

14. The method of claim 13, wherein the colonies are parent colonies, wherein the Mycobacteria of the first descendant generation are in descendant colonies, and wherein at least 30% of the descendant colonies are S-type colonies.

15. The method of claim 1, wherein the live Mycobacteria are attenuated.

16-20. (canceled)

21. The method of claim 1, wherein the Mycobacteria reside in the suspension fluid as a single cell suspension for a duration of less than two hours.

22. The method of claim 10, wherein the genome alteration causes the Mycobacteria to become auxotrophic.

23. The method of claim 1, wherein at least 50% of the Mycobacteria in the single cell suspension are dissociated into individual single cells.

24. The method of claim 1, wherein the Mycobacteria in the therapeutic composition have a trait for only S-type colony growth.

25. The method of claim 1, wherein the non-liquid culture medium is a solid or semi-solid culture medium.
